(19) United States
(12) Patent Application Publication
(10) Pub. No.: US 2007/0281998 A1
(43) Pub. Date: Dec. 6, 2007

(54) TREATMENT OF HOT FLASHES USING MUSCARINIC RECEPTOR ANTAGONISTS

(76) Inventor: Katherine D. LaGuardia, New York, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/755,907

(22) Filed: May 31, 2007

Related U.S. Application Data
(60) Provisional application No. 60/803,647, filed on Jun. 1, 2006.

(51) Int. Cl. A61K 31/24

(52) U.S. Cl. 514/540

(57) ABSTRACT

The present invention relates to a method of treating hot flashes by administering a muscarinic receptor antagonist. The method is useful for treating peri- and post-menopausal women, including women who have undergone surgically induced menopause.

### CROSS REFERENCE TO RELATED APPLICATIONS

This application claims the benefit of U.S. Provisional Application 60/803,647 filed on Jun. 1, 2006, which is incorporated by reference herein in its entirety.

### FIELD OF THE INVENTION

The present invention relates to the use of muscarinic receptor antagonists for the treatment of hot flashes (also referred to herein as hot flushes) in women. More particularly, the invention relates to the use of immediate and extended release formulations of these agents for the treatment of hot flashes in peri- and postmenopausal women.

### BACKGROUND OF THE INVENTION

Hot flashes are one of the most common health problems for peri- and postmenopausal women, affecting approximately 75% of this group. The vasomotor events characteristic of hot flashes typically begin 1 to 2 years prior to menopause and usually persist for 6 months to 5 years. Hot flashes are characterized by the sudden onset of intense warmth that begins in the chest and may progress to the neck and face. Hot flashes are often accompanied by anxiety, palpitations, profuse sweating and skin blotching. These symptoms can affect a woman's ability to work, her social life and sleep patterns and her general perception of health. Shanafelt, T., et al., “Pathophysiolgy and Treatment of Hot Flashes”, Mayo Clinic Proc. 2002; 77: 1207-1218.

The precise etiology of hot flashes in peri- and post-menopausal women is not known. However, it has been postulated that changes in estrogen levels at the time of menopause are primarily responsible for hot flashes. As a woman reaches menopause and exhausts her supply of ova there are no remaining follicles to secrete estrogen. Accordingly, the negative feedback exerted by the follicles on the hypothalamus and pituitary gland during the reproductive years is lost and pituitary gonadotropin production increases. Hot flashes typically occur simultaneously with these hormonal changes. While the specific mechanism by which a decrease in estrogen causes hot flashes is unknown, it appears that estrogen withdrawal, rather than low circulating estrogen levels, is the primary factor leading to hot flashes. Studies suggest that estrogen withdrawal affects thermoregulatory centers in the hypothalamus. These studies found that small changes in core body temperature occur 15 minutes before hot flashes in up to 60% of hot flash episodes. Women with hot flashes may have a thermoregulatory zone that is shifted downward and that is more narrow than women who do not have hot flashes. Since heat loss mechanisms can be triggered by as little as a 0.01° elevation of core body temperature above the regulatory zone, the subtle changes in temperature before a hot flash, coupled with a narrow homeostatic temperature zone, may trigger the heat loss mechanisms that lead to hot flash symptoms. Regulation of the thermoregulatory nucleus is governed by complex neuroendorcine pathways involving norepinephrine, estrogen, testosterone and endorphins, and these pathways are possible sites where dysfunction may occur in women who experience hot flashes. Shanafelt, T., et al., supra.

Estrogen replacement therapy (ERT) has traditionally been used for the treatment of hot flashes associated with menopause and has proven to be quite effective in relief of the associated symptoms. Although the benefits of ERT are well proven, the long-term use of unopposed estrogen in women who have a uterus is associated with an increase in the incidence of endometrial hyperplasia and the risk of endometrial cancer. The concomitant use of progestins in hormone replacement therapy (HRT) reduces this risk. However, research from the Women's Health Initiative, a long-term study sponsored by the National Institutes of Health, has demonstrated that the risks of HRT may outweigh the potential benefit. Such risks include significant increased risks of breast cancer, coronary heart disease, stroke and blood clots for some women on HRT.

Alternative therapies for hot flashes have been employed for many years. Prior to the commonplace use of ERT and HRT, women experiencing hot flashes were treated with antidepressants, sedatives and anticholinergic drugs. A trial involving Bellergal, a combination of ergotamine tartrate, belladonna alkaloids, and phenobarbital, versus placebo demonstrated a 60% reduction in hot flushes as compared to 22% with placebo. Lebherz T., et al., “Nonhormonal treatment of the menopausal syndrome: a double-blind evaluation of an autonomic system stabilizer”, Obstet Gynecol 1969;33:795-799. Another trial demonstrated a statistically significant reduction in climacteric complaints with Bellergal versus placebo after 2 and 4 weeks of therapy (no significant difference between the two groups was seen at 8 weeks). Bergmans M., et al, “Effect of Bellergal Retard on climacteric complaints: a double-blind, placebo-controlled study”Maturitas 1987;9(3):227-34.

Some of these therapies, such as treatment with older antidepressants and anticholenergic drugs, are considered to be obsolete. Rodstrom, K., et al., “A longitudinal study of the treatment of hot flushes: the population study of women in Gothenburg during a quarter century”, Menopause, Vol. 9, No. 3, pp 156-161 (2002). It is interesting to note that while treatment with older antidepressants is disfavored, newer antidepressants, in particular the selective serotonin reuptake inhibitors, have shown some promise in alleviating the symptoms associated with hot flashes. Studies have been carried out with several compounds in this class including paroxetine, fluoxetine and venlafaxine. Several small studies have also been conducted to test the efficancy of gabapentin, a γ-aminobutyric acid analogue, in the treatment of hot flashes. This compound is typically used in the treatment of neurological disorders such as epilepsy and neuropathic pain. Other therapies that have been investigated include clonidine, a centrally acting α-adrenergic agonist used to treat high blood pressure, and combinations of belladonna alkaloids and Phenobarbital. These latter combinations were widely used in the 1970s and 1980s; however, the small clinical benefit provided by these therapies was outweighed by the adverse effects of belladonna and the risk of phenobarbital dependence. Shanafelt, T., et al., supra.

It has surprisingly been found that while older anticholinergic and antimuscarinic agents are now disfavored for the treatment of hot flashes, newer muscarinic receptor antagonists can effectively treat hot flashes in women with only limited side effects.

### SUMMARY OF THE INVENTION

The present invention provides a method for treating hot flashes in peri- and postmenopausal women, including women who have undergone surgically induced menopause by administering a therapeutically effective amount of an muscarinic receptor antagonist. Muscarinic receptor antagonists useful in practicing the present invention include oxybutynin, tolterodine, trospium, darifenacin, solefenacin, hyoscyomine and combinations of these antagonists. Pharmaceutically effective salts or esters of these antaagonists may be utilized, and where the antagonist exists as a racemate, individual enanteomers or a racemaic mixture of the enanteomers may be employed. The muscarinic receptor antagonist may be provided either as an immediate release or extended release formulation and is administered via any route typically used for the administration of such agents, including oral, transdermal, topical, buccal, sublingual or microinjection administration.

Preferably, the muscarinic receptor antagonist is oxybutynin In the most preferred embodiment of the invention oxybutynin is administered via an extended release formulation, either orally or transdermally.

### BRIEF DESCRIPTION OF THE DRAWINGS

FIG. 1 illustrates the decrease in the frequency of hot flashes over a twelve week period in women being treated with extended release oxybutynin versus placebo.

FIG. 2 illustrates the decrease in the severity of hot flashes over a twelve week period in women being treated with extended release oxybutynin versus placebo.

### DETAILED DESCRIPTION OF THE INVENTION

A randomized, double blind, multi-center, parallel group, placebo-controlled study was conducted to evaluate the safety and efficacy of an extended release oxybutynin chloride formulation (Ditropan XL® available from Ortho-McNeil Pharmaceutical, Inc, Raritan, N.J.) for the treatment of hot flushes, in healthy naturally peri- and postmenopausal women. Subjects were randomized to Ditropan XL® 15 mg or placebo in a 1:1 ratio. The total duration of the study for each treatment group was approximately 98 days. Subjects were seen for their Pre-Randomization Visit (Visit 1) fourteen (14) days prior to randomization and a physical examination, medical history, hot flush history, vital signs and laboratory tests were performed. Subjects also had daily diaries dispensed to record their hot flushes (frequency for each severity). Subjects who meet the eligibility criteria for this study were randomized at Visit 2. At that visit, subjects had vital signs taken, adverse events recorded, study medication dispensed, and completed Quality of Life (QOL) questionnaires. Each subject was instructed to start her study medication beginning the morning after this visit (defined as Study Day 1). In both treatment groups, subjects returned for follow-up visits between Study Days 8-14 (Visit 3), 22-28 (Visit 4), and 50-56 (Visit 5). The Final Study Visit (Visit 6) occurred between Study Days 78-84.

Healthy females between the ages of 40 to 65 years were eligible to participate. Subjects must have experienced natural menopause, be symptomatic and experienced a mean of seven or more moderate to severe hot flushes with sweating per day, based upon data obtained from the subject's completed diary for 14 consecutive days between the pre-randomization visit and Visit 2 (pre-randomization period). Subjects were not eligible if they had a genitourinary condition that required the use of an anticholinergic agent. A total of approximately 140 females were recruited into the study (70 subjects in the Ditropan XL® group and 70 subjects in the placebo group). As noted above, subjects received either Ditropan XL® 15 mg or matching placebo. One tablet was taken orally every day in the morning for 12 weeks.

The primary endpoints in this study was the change in daily frequency of moderate to severe hot flushes from baseline to Week 12 (corresponding to visit 6 that was scheduled from day 78 to day 84) and the change in severity of moderate to severe hot flushes from baseline to Week 12. The baseline value for severity was defined as the result of adding the severity scores of moderate to severe hot flushes over the pre-randomization period (from visit 1 to visit 2) and dividing by the number of moderate to severe hot flushes during the corresponding period. The severity for Week 12 was defined as the result of adding severity scores of moderate to severe hot flushes over the last 7 days prior to last dose of study medication and dividing by the number of moderate to severe hot flushes during the corresponding period. The baseline value for daily frequency was defined as total number of moderate to severe hot flushes recorded during pre-randomization period divided by the number of days in the corresponding period for which complete diaries were received. The daily frequency for Week 12 was defined as the total number of moderate to severe hot flushes recorded during the last 7 days prior to last dose of study medication divided by the number of days in that week for which complete diaries were received

The secondary endpoints included change in daily frequency of moderate to severe hot flushes from baseline to Week 4, change in severity of moderate to severe hot flushes from baseline to Week 4, change of daily composite score of moderate to severe hot flushes from baseline to Week 4 and Week 12, change in daily frequency of any hot flushes from baseline to Week 4 and Week 12, change in severity of any hot flush from baseline to Week 4 and Week 12, and change in daily composite score of any hot flushes from baseline to Week 4 and Week 12. Other secondary endpoints included all scores from the Profile of Mood States, Pittsburgh Sleep Quality Index, Menopause-Specific Quality of Life Questionnaire, Short Form-36 Health Survey, and Sleep Disruption Scale, as well as the Subject Global Assessment score.

As noted above, subjects were dispensed a diary at the Pre-Randomization Visit (Visit 1) and started to record their hot flushes (frequency for each severity). The term hot flush is descriptive of a sudden onset of reddening of the skin over the head, neck, and chest, accompanied by a feeling of intense body heat and concluded by sometimes profuse perspiration. The duration varies from a few seconds to several minutes and, rarely, for an hour.

The severity of a hot flush was defined as:
1. Mild: sensation of heat without sweating
2. Moderate: sensation of heat with sweating, able to continue activity
3. Severe: sensation of heat with sweating, causing cessation of activity.

Waking episodes (i.e., episodes that wake the subject from sleep) associated with hot flushes were recorded separately and were considered severe.

The primary efficacy variables were the number and severity of hot flushes. The measurements of the QOL questionnaires were considered secondary evaluations. The Sleep Disruption Scale, Profile of Mood States, and Menopause-Specific Quality of Life questionnaires were administered at every visit starting at Visit 2. The Pittsburgh Sleep Quality Index and Short Form-36 Health Survey were administered at all double-blind treatment phase visits except Visit 3. At the Final Study Visit, each subject provided an overall evaluation of study treatment by completing a Subject Global Assessment. The rating scale for the final assessment by the subject included much better, better, slightly better, no meaningful difference, slightly worse, worse or much worse.

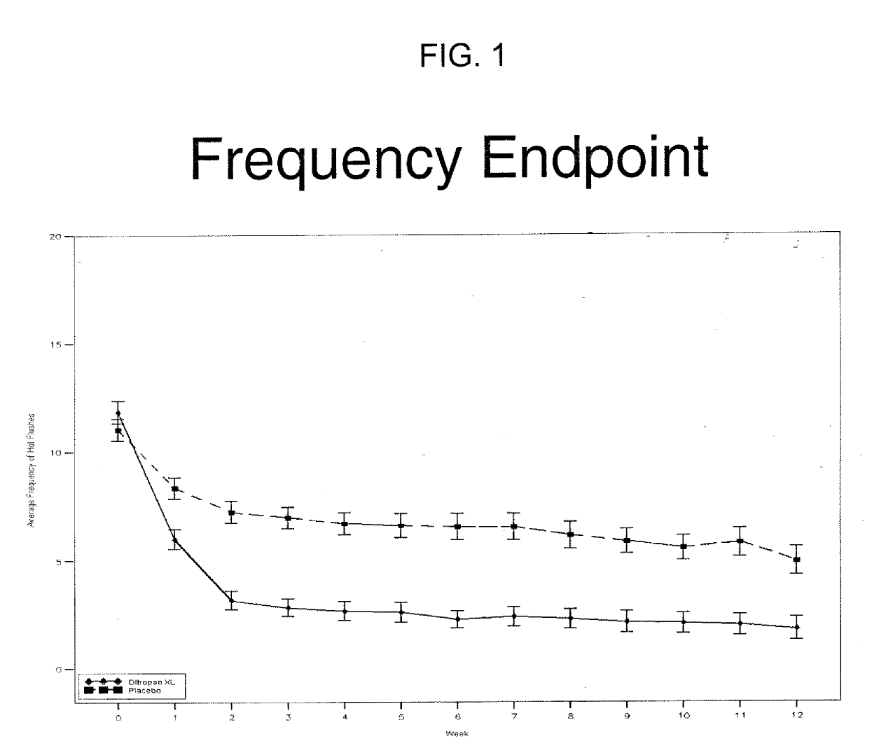

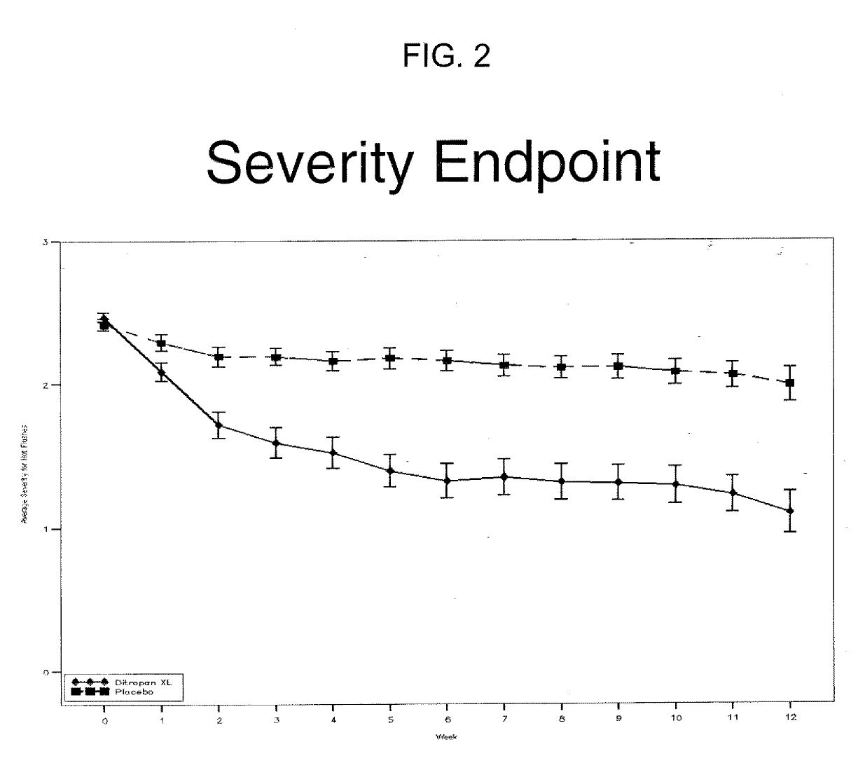

The results of the study are shown in Tables 1 and 2 and FIGS. 1 and 2. Table 1 shows the reduction in frequency of hot flashes obtained with DITROPAN XL over placebo over the 12 week trial as measured at baseline, week 4 and week 12.

TABLE 1
Fequency Endpoint - Baseline to Week 12

| | DXL (n = 72) | Placebo (n = 72) | P-value |
|---|---|---|---|
| Baseline (±SD) | 11.87 ± 4.436 | 10.84 ± 3.983 | |
| Week 4 (±SD) | 2.96 ± 3.640 | 6.78 ± 4.058 | <0.001 |
| Week 12 (±SD) | 2.38 ± 3.579 | 6.15 ± 5.546 | <0.001 |

The frequency endpoint data are shown graphically in FIG. 1. FIG. 1 illustrates that subjects taking DITROPAN XL not only achieved a statistically significant lowering in the frequency of hot flashes, but also a more rapid reduction in the frequency as compared to placebo.

Table 2 shows the reduction in severity of hot flashes obtained with DITROPAN XL over placebo over the 12 week trial as measured at baseline, week 4 and week 12.

TABLE 2
Severity Endpoint - Baseline to Week 12

| | DXL (n = 72) | Placebo (n = 72) | P-value |
|---|---|---|---|
| Baseline (±SD) | 2.47 ± 0.275 | 2.42 ± 0.356 | |
| Week 4 (±SD) | 1.56 ± 0.868 | 2.20 ± 0.513 | <0.001 |
| Week 12 (±SD) | 1.33 ± 0.987 | 2.10 ± 0.730 | <0.001 |

The severity endpoint data are shown graphically in FIG. 2. FIG. 2 illustrates that subjects taking DITROPAN XL not only achieved a statistically significant lowering in the severity of hot flashes, but also a more rapid reduction in the frequency as compared to placebo.

Various modifications of the invention in addition to those shown and described herein will be apparent to those skilled in the art from the foregoing description. Such modifications are also intended to fall within the scope of the appended claims. The cited patents or publications may provide further useful information and, accordingly, these cited materials are incorporated herein in their entirety by reference.

We claim:

1. A method for treating hot flashes in a subject in need of such treatment by administering a therapeutically effective amount of at least one muscarinic receptor antagonist, or a salt, ester, racemate or enanteomer of the antagonist.

2. The method of claim 1, wherein the subject is a peri- or post-menopausal woman or a women who has undergone surgically induced menopause.

3. The method of claim 1 wherein the muscarinic receptor antagonist is administered in an extended release formulation.

4. The method of claim 1, wherein the muscarinic receptor antagonist is oxybutynin.
